(19) United States
(12) Patent Application Publication
(10) Pub. No.: US 2024/0103004 A1
(43) Pub. Date: Mar. 28, 2024

(54) SYSTEMS AND COMPOSITIONS FOR DETECTING A BIOLOGICAL SAMPLE AND METHODS THEREOF

(71) Applicant: QCDx LLC, Farmington, CT (US)

(72) Inventors: Triantafyllos P. TAFAS, Rocky Hill, CT (US); Spencer Ryan KEILICH, Farmington, CT (US)

(21) Appl. No.: 18/477,825

(22) Filed: Sep. 29, 2023

Related U.S. Application Data
(63) Continuation of application No. PCT/US2022/022742, filed on Mar. 31, 2022.
(60) Provisional application No. 63/169,566, filed on Apr. 1, 2021.

(51) Int. Cl. G01N 33/58

(52) U.S. Cl. CPC G01N 33/582

(57) ABSTRACT

The present invention provides methods and systems for analyzing a biological sample. The methods of the present invention can utilize a labeling moiety and a cleavage moiety. The labeling moiety can comprise a binding moiety that is coupled to a detectable tag via a polynucleotide linker. The cleavage moiety can form a complex with the polynucleotide linker. In some cases, after formation of the complex, the cleavage moiety can effect cleavage of the polynucleotide linker, to release the detectable tag from the binding moiety. The release of the detectable tag can allow one or more additional imaging of the biological sample with a different labeling moiety that comprises a different binding moiety but the same labeling moiety.

### CROSS-REFERENCE

This application is a continuation of International Patent Application No. PCT/US2022/022742, filed Mar. 31, 2022, which claims the benefit of U.S. Provisional Patent Application No. 63/169,566 filed on Apr. 1, 2021, each of which is incorporated herein by reference in its entirety.

### BACKGROUND

Detection and analysis of target cells can be valuable for various applications, e.g., clinical and therapeutic applications. In some cases, early stage and even small tumors can release cancer cells in blood that carry a signature in the form of circulating tumor cells (CTCs) and can be responsible for the creation of metastases. Thus, cancer management can require frequent monitoring over time, including multiple repeat biopsies and analysis of one or more cells in the biopsies.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some embodiments, the disclosure provides a method for analyzing a biological sample, the method comprising:
(a) contacting the biological sample with a labeling moiety, wherein the labeling moiety comprises a binding moiety that is coupled to a detectable tag via a polynucleotide linker, wherein the binding moiety exhibits specific binding to a target ligand;
(b) subsequent to (a), imaging the biological sample to obtain an image, wherein the image is indicative of presence or absence of the target ligand in the biological sample based on staining or lack of staining by the labeling moiety; and
(c) subsequent to (b), contacting the biological sample with a cleavage moiety, wherein the cleavage moiety forms a complex with the polynucleotide linker, wherein, after formation of the complex, the cleavage moiety effects cleavage of the polynucleotide linker to release the detectable tag from the binding moiety.

In some embodiments, the disclosure provides a system for analyzing a biological sample, the system comprising:
a chamber, wherein the chamber comprises a container for holding the biological sample;
an imaging unit optically coupled to the chamber, wherein the imaging unit is configured to image the biological sample when disposed in the container; and
a processor operatively coupled to the imaging unit, wherein the processor is configured to:
   (a) direct flow of a labeling moiety to the container, wherein the labeling moiety comprises a binding moiety that is coupled to a detectable tag via a polynucleotide linker, wherein the binding moiety exhibits specific binding to a target ligand;
   (b) subsequent to (a), direct the imaging unit to image the biological sample in the container, to obtain an image, wherein the image is indicative of presence or absence of the target ligand in the biological sample based on staining or lack of staining by the labeling moiety; and
   (c) subsequent to (b), direct flow of a cleavage moiety to the container, wherein the cleavage moiety forms a complex with the polynucleotide linker, wherein, after formation of the complex, the cleavage moiety effects cleavage of the polynucleotide linker to release the detectable tag from the binding moiety.

### BRIEF DESCRIPTION OF THE DRAWINGS

FIG. 1 schematically illustrates an example process of generating a labeling moiety.

FIG. 2 schematically illustrates an example process of analyzing a biological sample with a labeling moiety and a cleavage moiety.

FIG. 3 shows a drawing for the sample holder and of the sample handler of the present invention.

FIG. 4 schematically illustrates Fluorescence Light Sheet Microscopy Principle for imaing, e.g., three-dimensional (3D) optimal tomography.

### DETAILED DESCRIPTION

Conventional immunohistochemistry (IHC) is a widely used diagnostic technique in tissue pathology. However, such conventional IHC can be associated with a number of limitations, including the limited number of markers that can be detected per tissue section or per imaing. In some cases, the number of markers that can be detected is limited by the number of available detectable tags (e.g., dyes) with different excitation and emission wavelengths. Thus, an unmet need exists for methods and systems that allow detection of a panel of markers (e.g., greater than 10, greater than 20, greater than 30, etc.) on the same biological sample, with a limited number of detectable dyes, e.g., via targeted and specific deactivation (e.g., cleavage) of the detactable tags from secondary antibodies after a cycle of (a1) staining of target antigens via primary antibodies and (a2) staining of primary antibodies with secondary antibodies conjugated to the detectable tags, or after a cycle of (b) staining of target antigens via primary antibodies that are directly conjugated with detectable tags.

Methods and Systems for Analyzing a Biological Sample

Described herein is a multiplexing technology (e.g., multiplexing of labeling moieties, such as immunofluorescent probes) for use on live or fixed cells, in which the cells can be visualized intact in immobilized suspensions. Further described herein is microscope that can visualize a plurality of signals (e.g., at least or up to about 6 fluorescent signals) in a given cell suspension. To visualize a plurality of markers in the same cells, multiplex rounds of staining by detectable tags (e.g., fluorescent staining) can be required. The probe technology described herein can be designed to accommodate such repeated staining rounds on a biological sample, e.g., both live and fixed cells. The probes described herein can be used for multiplex staining of cell smears or thin tissue sections on traditional microscope slides.

In immunofluorescence microscopy, the number of antibodies interrogated can be restricted by the fluorescence channels used by the particular microscope. The broad emission spectra of fluorochromes can allow discrimination of about 6 fluorescence channels for a given cell preparation. Thus, multiplex immunofluorescence technology can be used for biopsied thin tissue sections allow for stripping the initial set of probes from a slide preparation and the applying a new set of probes. This technique can allow for highly multiplexed tissue imaging technologies and allow comprehensive studies of cell composition, functional state, and cell-cell interactions, which can have an improved diagnostic benefit. These imaging techniques can use, for example, cyclic immunofluorescence, tyramide-based mIHC/IF, epitope-targeted mass spectrometry, or RNA detection.

In cancer diagnosis, solid tumor biopsies are used to identify the mutational profile in lesion locations. Reporting on ongoing changes of tumor heterogeneity is difficult using longitudinal biopsies of solid tissues. Liquid biopsies of circulating tumor DNA (ctDNA) or circulating tumor cells (CTC) can be used for elucidating the advancing disease heterogeneity and acquired resistance to treatment. Analysis of CTCs, CTC clusters, and immune cells can be used to analyze the tumor's changing molecular compositions, as real-time liquid biopsy. Multiplex imaging methods are important for revealing both CTCs and immune profiles heterogeneity. A variety of approaches can be used including cyclic imaging of successive fluorescent staining, antibody-DNA barcoding, imaging mass cytometry by time-of-flight, and mRNA in situ hybridization.

For clinical applications and given the rarity of CTCs in a patient sample, the multiplex staining method described herein can provide detailed molecular characterization for numerous phenotypic and treatment biomarkers for analysis of both live and fixed cells. The multiplex staining process described herein can protect the cellular integrity so that single CTC or immune cell isolation is possible for downstream single-cell molecular investigations.

The multiplex staining system described herein can allow for repeated rounds of staining with fluorescently labeled antibodies (e.g., with a limited number of fluorescent labels) on immobilized, live, or fixed cells. The probes disclosed herein can use a DNA oligomer as a linker to connect selected fluorochromes with monoclonal antibodies (e.g., primary antibodies that directly target and bind target antigens). The DNA oligomer sequence can then be programmed into a reagent comprising a polynucleotide-targeting agent (e.g., a cleavage moiety, including an endonuclease, such as a CRISPR-Cas protein). For example, a cell preparation can be stained with a probe disclosed herein and imaged with a microscope disclosed herein to record a first round of fluorescent signals and detect target cells. Then the preparation can be contacted with the CRISPR-Cas (e.g., CRISPR-Cas9) reagent that can cleave the linker (e.g., a polynucleotide linker that couples the fluorochome to the antibody), release the fluorochromes, and allow for a second round of staining. Previously identified cells of interest can then be visited in a rapid fashion to assess expression of antigens targeted by the second or subsequent rounds of fluorescent antibodies.

In a system disclosed herein, cells are visualized in 3D immobilized preparations, which can be perfused with media at the microliter level via an automated microfluidic system. The probes described herein can be designed to be used with an automated microscope system describe herein. In some embodiments, a biological sample (e.g., the immobilized cell suspension) can be contacted with a first set of labeling moieties (e.g., 6 immunofluorescent primary antibody probes) via automated perfusion with reagent solutions, e.g., in accordance with the cell staining protocol described herein. Subsequently, the biological sample can be imaged to detect presence or absence of the first set of labeling moieties (e.g., the entire suspension of fluorescently stained cells can be imaged), e.g., to identify one or more target cells along with recordation of their respective three-dimensional (3D) position within the biological sample. At a next step, the biological sample (e.g., the cell suspension) can be perfused with reagent solutions applying a cleavage moiety protocol (e.g., a CRISPR-Cas protocol). For example, the CRISPR-Cas system (e.g., CRISPR-Cas9) system comprises the Cas endonuclease (e.g., Cas9 protein) and a guide nucleic acid molecule (e.g., a small guide RNA or sgRNA). The guide nucleic acid molecule has two molecular components (e.g., as two separate nucleic acid molecules, or within a single nucleic acid molecule): a CRISPR RNA (crRNA), which is specific to a genomic locus of the that is complementary to the target gene of interest (e.g., DNA oligomer of interest), and an auxiliary trans-activating crRNA (tracrRNA). Guided by the guide nucleic acid molecule, the Cas endonuclease (e.g., Cas9) protein can specifically recognize the genomic locus and can cleave the linker. Cleavage can lead to specific release of the fluorescent signals from the target cells. The target cells can then be available for a next staining round of immunofluorescent staining that targets a different set of cellular antigens (e.g., via an additional set of one or more labeling moieties as disclosed herein).

In some embodiments, the methods and systems disclosed herein (e.g., via use of the one or more cleavage moieties, such as the CRISPR/Cas9 system) can be fully compatible with live cells and allows for staining of live cell suspensions for ex vivo detection of cell surface markers. The methods and systems disclosed herein can also allow detection of intracellular proteins following fixation and permeabilization of the suspended cells.

In some embodiments, the polynucleotide-targeting agent as disclosed herein is present or active in the extracellular portion of a target cell (e.g., a live target cell) to be imaged. In some embodiments, the polynucleotide-targeting agent is present or active in the intracellular portion of the target cell (e.g., a permeabilized, fixed, and/or immobilized target cell). In some embodiments, the polynucleotide-targeting agent is not expressed or released by the target cell. In some embodiments, the target cell is not engineered to express the polynucleotide-targeting agent.

In some embodiments, the system and method of the present disclosure can allow detection (e.g., automated detection without human intervention) of a plurality of target antigens (different target antigens) using at least or up to about 1 optical channel (e.g., fluorescence channel), at least or up to about 2 optical channels (e.g., different fluorescence channels), at least or up to about 3 optical channels, at least or up to about 4 optical channels, at least or up to about 5 optical channels, at least or up to about 6 optical channels, at least or up to about 7 optical channels, at least or up to about 8 optical channels, at least or up to about 9 optical channels, at least or up to about 10 optical channels, or at least or up to about 15 optical channels. The plurality of target antigens can comprise at least or up to about 2 antigens, at least or up to about 3 antigens, at least or up to about 4 antigens, at least or up to about 5 antigens, at least or up to about 6 antigens, at least or up to about 7 antigens, at least or up to about 8 antigens, at least or up to about 9 antigens, at least or up to about 10 antigens, at least or up to about 15 antigens, at least or up to about 20 antigens, at least or up to about 30 antigens, at least or up to about 40 antigens, at least or up to about 50 antigens, at least or up to about 60 antigens, at least or up to about 70 antigens, at least or up to about 80 antigens, at least or up to about 90 antigens, at least or up to about 100 antigens, at least or up to about 200 antigens, or at least or up to about 500 antigens. In some embodiments, the term detection can comprise determining a presence of the target antigen(s) or lack thereof.

In some embodiments, an antibody as disclosed herein can be a proteinaceous binding molecule with immunoglobulin-like functions. Monoclonal and polyclonal antibodies, and derivatives, variants, and fragments thereof are contemplated. Non-limiting examples of antibodies include immunoglobulins (Ig's) of different classes (i.e. IgA, IgG, IgM, IgD and IgE) and subclasses (such as IgG1, IgG2, etc.). A derivative, variant, or fragment thereof can be a functional derivative or fragment that retains the binding specificity (e.g., complete and/or partial) of the corresponding antibody. Antigen-binding fragments include Fab, Fab′, F(ab′)2, variable fragment (Fv), single chain variable fragment (scFv), minibodies, diabodies, and single-domain antibodies (sdAb, nanobodies, or camelids). Antibodies and fragments thereof can be optimized, engineered, or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

In some embodiments, a polynucleotide-targeting agent as disclosed herein can be heterologous (e.g., a heterologous polypeptide, such as a heterologous nuclease or endonuclease) to the biological sample (e.g., to one or more target cell(s) in the biological sample or derived from the biological sample) to be imaged by the systems and methods of the present disclosure. The polynucleotide-targeting agent can be configured to specifically bind to (or complex with) a target polynucleotide sequence. Non-limiting examples of the polynucleotide-targeting agent can include a CRISPR-associated polypeptide (Cas), zinc finger nuclease (ZFN), zinc finger associate gene regulation polypeptides, transcription activator-like effector nuclease (TALEN), transcription activator-like effector associated gene regulation polypeptides, meganuclease, natural master transcription factors, epigenetic modifying enzymes, recombinase, flippase, transposase, RNA-binding proteins (RBP), an Argonaute protein, any derivative thereof, any variant thereof, or any fragment thereof. In some embodiments, the polynucleotide-targeting agent can comprise (e.g., innately comprise) cleavage activity against at least a portion of the target polynucleotide sequence (e.g., to release a probe, such as a fluorescent probe, from the target polynucleotide sequence). Alternatively or in addition to, the polynucleotide-targeting agent can be operatively coupled to (e.g., directly fused with or indirectly coupled to) a cleavage moiety (e.g., a separate nuclease) to cleave at least a portion of the target polynucleotide sequence.

In some embodiments, a cleavage moiety can comprise CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides. Non-limiting examples of Cas proteins can include c2c1, C2c2, c2c3, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Cas9 (Csn1 or Csx12), Cas10, Cas10d, Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cul966, and modified variants thereof.

In some embodiments, the cleavage moiety (e.g., a CRISPR/Cas endonuclease and a guide nucleic acid molecule) can be introduced or delivered to the biological sample (e.g., to the extracellular portion of one or more target cells in the biological sample) without a solid carrier (e.g., a viral capsule, or a non-viral delivery moieties). For example, the cleavage moiety can be suspended in a solution (e.g., in a buffer). In some embodiments, the cleavage moiety can be introduced or delivered to the biological sample via a non-viral delivery moieties, such as, for example, virosomes, liposomes, immunoliposomes, exosomes, nanoparticles, microparticles, etc.

In some embodiments, the labeling moiety can comprise a single binding moiety (e.g., an antibody). For example, a primary antibody that exhibits specific binding to a target ligand/antigen can be directly functionalized with a detactable tag (e.g., a fluorophore) via a polynucleotide linker as disclosed herein. In some embodiments, the labeling moiety can comprise a plurality of binding moieties (e.g., a plurality of antibodies). For example, a primary antibody is not functionalized with a detectable tag. Instead, a secondary antibody that exhibits specific binding to the primary antibody can be functionalized with a detactable tag (e.g., a fluorophore) via a polynucleotide linker as disclosed herein.

In some embodiments, the target polynucleotide can have a length of at least or up to about 5 nucleobases, at least or up to about 10 nucleobases, at least or up to about 15 nucleobases, at least or up to about 20 nucleobases, at least or up to about 25 nucleobases, at least or up to about 30 nucleobases, at least or up to about 35 nucleobases, at least or up to about 40 nucleobases, at least or up to about 45 nucleobases, at least or up to about 50 nucleobases, at least or up to about 60 nucleobases, at least or up to about 70 nucleobases, at least or up to about 80 nucleobases, at least or up to about 90 nucleobases, or at least or up to about 100 nucleobases, at least or up to about 110 nucleobases, at least or up to about 120 nucleobases, at least or up to about 130 nucleobases, at least or up to about 140 nucleobases, at least or up to about 150 nucleobases, at least or up to about 160 nucleobases, at least or up to about 170 nucleobases, at least or up to about 180 nucleobases, at least or up to about 190 nucleobases, at least or up to about 200 nucleobases, at least or up to about 250 nucleobases, at least or up to about 300 nucleobases, at least or up to about 400 nucleobases, or at least or up to about 500 nucleobases.

In some embodiments, the target polynucleotide can be a single-stranded nucleic acid molecule (e.g., a single-stranded DNA or a single-stranded RNA. In some embodiments, the target polynucleotide can be a double-stranded nucleic acid molecule (e.g., a double-stranded DNA or a double-stranded RNA.

In some embodiments, contacting the biological sample with the cleavage moiety as disclosed herein is not and need not comprise expressing the cleavage moiety from one or more target cells of the biological sample. For example, the cleavage moiety can comprise a polypeptide and/or a polynucleotide (e.g., an endonuclease such as a CRISPR Cas protein and a respective guide nucleic acid molecule), and the recombinant forms of the polypeptide and/or the polynucleotide (e.g., expressed and/or purified elsewhere) are introduced to the biological sample for imaging/analyzing the biological sample.

In some embodiments, the contacting the biological sample with a labeling moiety can comprise contacting the biological sample with a plurality of labeling moieties, wherein each labeling moiety of the plurality of labeling moieties comprises (i) a unique binding moiety that exhibits specific binding to a unique target ligand and (ii) a unique detectable tag that is coupled to the unique binding moiety via a unique polynucleotide linker.

In some embodiments, the system as disclosed herein can comprise a chamber, an imaging unit, and a processor. In some embodiments, the chamber comprises a container for holding the biological sample. In some embodiments, the imaging unit is optically coupled to the chamber. The imaging unit can be configured to image the biological sample when disposed in the container. In some embodiments, the processor is operatively coupled to (i) the chamber and/or (ii) the imaging unit. The processor can be configured to perform one or more steps of the methods disclosed herein.

In some embodiments, the system can further comprise one or more reservoirs (e.g., a plurality of reservoirs). The reservoir(s) can be utilized to store one or more reagents used in the methods disclosed herein. Each reservoir can be in fluid communication with at least the chamber (e.g., the container), such that one or more reagents from the reservoir can be directed to be transferred (e.g., directed to flow) from the reservoir and towards and into the chamber. For example, a reservoir can be a source of one or more labeling moieties as disclosed herein. In another example, a reservoir can be a source of one or more cleavage moieties as disclosed herein.

In some embodiments, the methods disclosed herein do not and need not comprise deactivation (e.g., bleaching) of the detectable tags (e.g., fluoropores). Cleavage of the polypeptide linker and the resulting release of the polypeptide linker from the binding moiety are sufficient to remove substantially all of the detactable tags (or substantially all of a detectable level of the detectable tags) from the biological sample.

In some embodiments, the methods disclosed herein do not and need not utilize electromagnetic energy or ion beams to effect release of the detectable tags (e.g., fluoropores) from the binding moieties. Use of the cleavage moiety (e.g., comprising an enzyme, such as a CRISPR/Cas protein) can be sufficient to remove substantially all of the detactable tags (or substantially all of a detectable level of the detectable tags) from the biological sample.

In some embodiments, the present disclosure provides a system comprising (i) a polynucleotide linker that is coupled to a detectable tag and (ii) a cleavage moiety. The polynucleotide linker can be usable for functionalizing a binding moiety (e.g., an antibody), e.g., to generate a binding moiety as described herein. As described herein, the cleavage moiety can be capable of forming a complex with the polynucleotide linker, such that, upon formation of the complex, the cleavage moiety can effect cleavage of the polynucleotide linker, to release the detectable tag from the binding moiety. The polynucleotide linker and the cleavage moiety can be provided in separate compositions. The system can comprise at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 11, at least or up to about 12, at least or up to about 13, at least or up to about 14, at least or up to about 15, or at least or up to about 20 polynucleotide linkers. The cleavage moiety can comprise a CRISPR Cas endonuclease and a guide nucleic acid molecule. The system can comprise at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 11, at least or up to about 12, at least or up to about 13, at least or up to about 14, at least or up to about 15, or at least or up to about 20 guide nucleic acid molecules, wherein each guide nucleic acid molecule exhibits binding affinity to its unique target polypeptide sequence (e.g., its unique target polynucleotide linker).

Additional Details of Imaging Systems and Methods

The systems and methods as disclosed herein can permit ex vivo observation of cells (e.g., cells that have been stained with vital stains for CTC-specific biomarkers and maintained alive for periods of time) supported by a three-dimensional (3D) culture subsystem. In some embodiments, the system can comprise a biological holder and a handler. A specially designed cell chamber can be fitted for input and output of culture media, gas regulation and control of environmental variables (temperature, pH etc). This arrangement can allow ex vivo observation of cells while perfused with culture media which may contain various substances. The chamber can be fitted with a micromanipulator (handler) used to isolate target cells under direct observation. Both the chamber and the micromanipulator can be operated automatically by a system computer and software system.

The ex vivo liquid biopsy can offer longitudinal observation of target cells, e.g. CTCs and/or white blood cells (WBCs) and assessment of desired and undesired toxicity of therapeutic drug cocktails before used for patient treatment. This provision can drive precision medicine for improved outcomes and reduced adverse effects to the patient. Cell isolation can allow CTC genomic and transcriptomic analysis that can reveal improved therapeutic options, tuned to the patient's current disease status.

The sample holder and handler of the present invention, combined with deep quantitation of every cell the specimen, can be a precision medicine tool. Deep CTC characterization and single-cell, genomic/transcriptomic analysis can allow that the oncologist select a treatment that is synchronized with the current disease stage. Ex vivo assessment of how a selected drug or drug combination affects CTCs and/or WBCs in the patient's blood can be assessed in view of patient outcomes.

In some embodiments, a central computer system operates a software package that (a) acquires and processes images of the biological specimen's features for identification and quantitation, (b) actuates the motorized components, pumps, sensors of the system, (c) operates a robotic arm that loads and unloads samples, and (d) handles digital information managed in local or wide area networks. The central computer system may utilize local or distributed processing protocols.

The system also includes or is coupled to a tunable laser source or multiple single wavelength laser sources, complete with light management optical path(s). An optical system modulating the light (e.g., light sheet, such as laser light sheet) can combine bilateral illumination to produce the sheet illumination for Selective Plane Illumination Microscopy (SPIM).

In some embodiments, imaging is performed by illuminating the specimen with narrow spectrum excitation light provided by monochromatic and/or tunable laser sources. Images of the resulting emission are acquired by high sensitivity monochrome cameras on a field by field basis. These images are combined in 3D stacks, which are then analyzed for quantitative measurement of biomarker levels in the individual cells. Alternatively, the images can be analyzed individually (e.g., without combining multiple images into a single image).

In operation, a biological specimen that can include live cells is stained with a variety of markers against proteins, nucleic acids, or other cellular components and encased in an appropriately shaped cylindrical sheath to be fitted on a biological sample holder. The preparation is made by mixing the cell suspension with a solid or semi-solid medium (e.g., gels, such as agarose or other hydrogels that are compatible with preserving the subcellular structure of the embedded cells), at a temperature where the solution is still liquid. In addition to the cells, fluorescent beads that act as fiducial reference for the identified cells are added to the solution. The liquid cell/bead/gel suspension is aspirated in tubing chosen to be transparent to the fluorescence light regime utilized. After being allowed to solidify, the specimen can be visualized in the light path. The biological specimen is mounted on a specimen holder loaded onto the microscope stage.

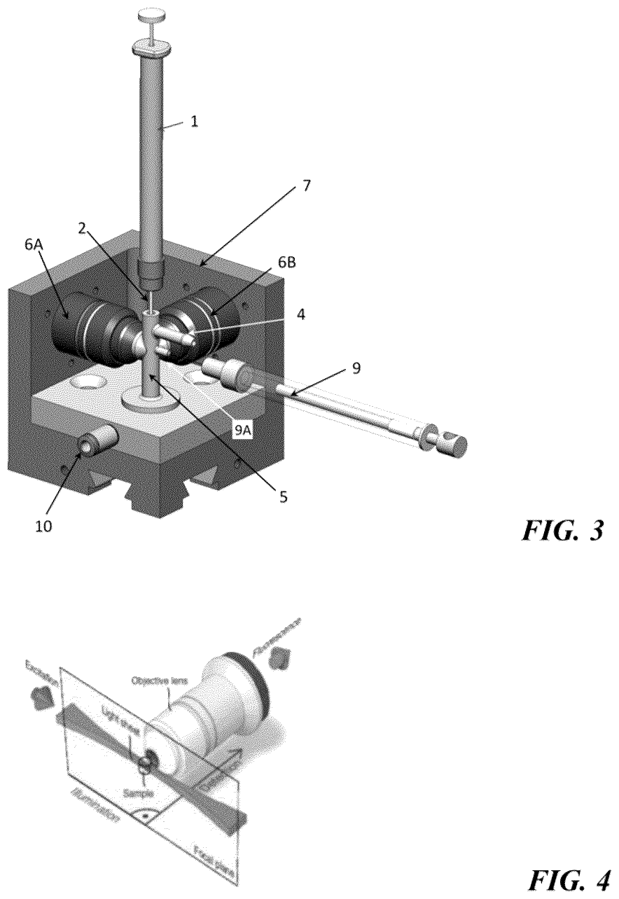

FIG. 3 shows a drawing for the sample holder and of the sample handler of the present invention. Shown is a component 1 for advancing and manipulating the sample 3 (not visible in this FIG. 3) contained within a sample holder such as a capillary tube 2 with a plurality of holes 2A (the capillary tube is not visible in this FIG. 3. The component 1 can be any of a variety of mechanical device, including, for example a glass syringe. Shown is the cylindrical sample chamber 5, with a fluid output or outlet port 4, a lens holder 7, holding an illumination lens 6A, and a detection lens 6B (which are oriented orthogonally or perpendicularly, e.g., at 90 degrees to each other), and an access port 9A built into the cylindrical sample chamber 5 for allowing access for a device for retrieving particles of interest, such as a micropipette 9. A fluid input connector 10 is shown on the base of the lens holder 7. Not visible is the fluid input orifice, of the cylindrical sample chamber 5 located in the base of the chamber. In further embodiments, the component for advancing the sample can be controlled by an external motor, such as a 4-D motor 13 (not shown in FIG. 3) to provide movement and control in the X, Y, and Z axes, and to provide for rotation of the sample. The optical axes of the lenses 6A and 6B can be orthogonal and co-planar such that the sample chamber and sample can be positioned at the intersection of the respective optical axes for the lenses.

The system as disclosed herein (e.g., the RareScope system) utilized Fluorescence Light Sheet Microscopy to analyze intact, stained cells immobilized in hydrogel with a 3D optical tomographic approach (FIG. 4). In some embodiments, a cell suspension can be observed in SPIM instrument mounted in fixture and embedded in hydrogels that allow cell perfusion with fluorescently labeled antibodies, fluorescence in situ hybridization immunostaining, and/or fluorescence in situ hybridization (FISH) probes, and other stains and media that can sustain ex vivo cell observation.

Light sheet fluorescence microscopy (LSFM) is a fluorescence microscopy technique in which a sample is illuminated by a laser light sheet (i.e. a laser beam focused in only one direction) perpendicularly (e.g., orthogonally or 90 degrees to the direction of observation). The light sheet can be created using, for example, cylindrical lens or by a circular beam scanned in one direction to create the light sheet. In LSFM, only the observed section of a sample can be illuminated. Therefore, LSFM can reduce the photodamage and stress induced on a living sample. In addition, good optical sectioning capability of LSFM can reduce the background signal, and thus can create images with higher contrast, comparable to confocal microscopy. Fluorescence light-sheet microscopy can bridge the gap in image quality between fluorescence stereomicroscopy and high-resolution imaging of fixed tissue sections. Furthermore, high depth penetration, low bleaching, and/or high acquisition speeds can make light-sheet microscopy ideally suited for extended time-lapse experiments.

In some embodiments, the following steps are performed: Compare performance of embedding gels including agarose, collagen, polyacrylamide and tubing such as micro-perforated, fluorinated polyethylene (FPE) and glass both for fixed and live cells. Optimize fixation/permeabilization protocols. Assess need of antifading for fluorescence bleaching. Adapt SPIM image acquisition to materials chosen. Quantitative analysis of cell staining and identification or analysis of CTCs (e.g., via 3D image analysis and/or multiple antigen staining as disclosed herein).

In some embodiments, the present invention can comprise instruments and kits for the detection and characterization of CTCs and other target cell populations.

In some embodiments, a sample of cells (e.g., comprising at least one cell) of the biological sample can be analyzed by the systems and methods of the present disclosure. In some embodiments, at least one cell from a biological sample obtained from a subject can be analyzed by the systems and methods of the present invention. The biological sample can be a liquid sample, such as blood. The at least one cell can comprise at least or up to about 1 cell, at least or up to about 2 cells, at least or up to about 5 cells, at least or up to about 10 cells, at least or up to about 20 cells, at least or up to about 50 cells, at least or up to about 100 cells, at least or up to about 200 cells, at least or up to about 500 cells, at least or up to about 1000 cells, or more.

In some embodiments, the cell of the biological sample can be stained with a detection moiety (e.g., a plurality of detection moieties). The detection moiety can be capable of binding to a ligand of the cell. The ligand can be an extracellular ligand, a membrane-bound ligand, or an intracellular ligand. The ligand can be a small molecule, a polypeptide (e.g., a peptide or a protein), or a polynucleotide (e.g., ribonucleic acid (RNA), mRNA, deoxyribonucleic acid (DNA), etc.). The detection moiety can be an antibody, a monoclonal antibody, a polyclonal antibody, a recombinant antibody, a human antibody, a humanized antibody, a Fab, a Fab′, a F(ab′)2, an Fv, a single chain antibody (e.g., scFv), a minibody, a diabody, a single-domain antibody (sdAb, nanobodies, or camelids), or an Fc binding domain. In some embodiments, the cell can be treated with the detection moiety prior to being immobilized in the sample holder as disclosed herein. Alternatively or additionally, the cell can be treated with the detection moiety subsequent to being immobilized in the sample holder.

In some embodiments, the detection moiety can comprise a plurality of detection moieties that are different (e.g., multiplexing with multiple antibodies). The plurality of detection moieties can comprise at least or up to about 2 detection moieties, at least or up to about 3 detection moieties, at least or up to about 4 detection moieties, at least or up to about 5 detection moieties, at least or up to about 6 detection moieties, at least or up to about 7 detection moieties, at least or up to about 8 detection moieties, at least or up to about 9 detection moieties, at least or up to about 10 detection moieties, at least or up to about 15 detection moieties, or at least or up to about 20 detection moieties. The plurality of detection moieties can target different ligands.

In some embodiments, the plurality of detection moieties can bind a plurality of ligands that are indicative of different cell functions or cell states (e.g., different cell types, different cell origins, etc.). For examples, the plurality of ligands can be indicative different stages of cellular differentiation (or dedifferentiation). The plurality of detection moieties can comprise (i) a first detection moiety exhibiting specific binding to a first target ligand, wherein the first target ligand is a marker of a first cell type, and (ii) a second detection moiety exhibiting specific binding to a second target ligand, wherein the second target ligand is a marker for a second cell type that is different from the first cell type.

In some embodiments, different cell states (e.g., different cell types) can comprise stem cells and/or differentiated cells. Non-limiting examples of different cell types (e.g., including stem cells and/or differentiated cells) can include lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Endothelial cell, Vascular smooth muscle cell, Lymphatic endothelial cell, Atherosclerosis cell, Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells. Non-limiting examples of stem cells can include adult stem cells (e.g., mesenchymal stem cells), embdyonic stem cells, induced pluripotent stem cells, and progenitor cells (e.g., cardiac progenitor cells, neural progenitor cells, etc.).

In some embodiments, the first cell type as disclosed herein can be a differentiated cell type, such as an epithelial cell. The first ligand can comprise an epithelial cell antigen, such as epithelial cellular adhesion molecule (EpCAM) or cytokeratin (CK). In some examples, the first ligand can be one of EpCAM and CK, and the other antigen of EpCAM and CK can be bound and detected by a third detection moiety exhibiting specific binding to the other antigen. Non-limiting examples of the epithelial cell maker can include EpCam, Cadherin, Mucin-1, Cytokeratin (CK) 8, epidermal growth factor receptor (EGFR), cytokeratin (CK)19, ErbB2, PDGF, L6, Trop2, and leukocyte associated receptor (LAR).

In some embodiments, the second cell type as disclosed herein can be a stem cell type, such as a mesenchymal cell (e.g., mesenchymal stem cell). The second ligand can comprise a mesenchymal steat antigen, such as vimentin (Vim). Non-limiting examples of mesenchymal cell marker can include CD90, CD73, CD44, and vimentin.

In some embodiments, the cell as disclosed herein can be detected to exhibit only one of the plurality of ligands, and such characteristic can be indicative of the cell being a CTC. In some embodiments, the at least one cell as disclosed herein can be detected to exhibit two or more of the plurality of ligands, and such characteristic can be indicative of the at least one cell being a CTC. In some embodiments, a CTC from the sample of cells is determined to have been detected when (i) a number of cells determined to exhibit two or more of the plurality of ligands is greater than or equal to (ii) a number of cells determined to exhibit only one of the two or more of the plurality of ligands. For example, a CTC associated with breast cancer can be determined to have been detected from the sample of cells when (i) a number of cells determined to exhibit two or more of the plurality of ligands (e.g., EpCAM and Vim) is greater than or equal to (ii) a number of cells determined to exhibit only one of the two or more of the plurality of ligands (e.g., EpCAM substantially alone, or Vim substantially alone).

In some embodiments, the method disclosed herein can identify different types of diseased cells. In some embodiments, the method disclosed herein can assess heterogeneity within a specific population of diseased cells. In some embodiments, the specific population of diseased cells can be CTCs, and the method disclosed herein can assess heterogeneity (e.g., different subtypes or phenotypes) within the specific population of the CTCs. In some embodiments, the method disclosed herein can assess different phenotypes or states of a population of CTCs from breast tumors. For example, the method disclosed herein can identify, distinguish, and/or quantitate (i) CTCs of mesenchymal phenotype and/or (ii) CTCs of epithelial phenotype. In another example, the method disclosed herein can identify distinguish, and/or quantitate (i) CTCs of Luminal A breast cancer, (ii) CTCs of Luminal B breast cancer, (iii) CTCs of triple-negative breast cancer, (iv) CTCs of HER2-enriched breast cancer, and/or (v) CTCs of normal-like breast cancer.

CTCs of Luminal A breast cancer can be hormone-receptor positive (e.g., estrogen-receptor and/or progesterone-receptor positive), HER2 negative, and with low levels of the protein Ki-67. CTCs of Luminal B breast cancer can be hormone-receptor positive (e.g., estrogen-receptor and/or progesterone-receptor positive), either HER2-positive or HER2-negative, and with high levels of Ki-67. CTCs of triple-negative breast cancer can be hormone-receptor negative (e.g., estrogen-receptor and progesterone-receptor negative) and HER2 negative. CTCs of HER2-enriched breast cancer can be hormone-receptor negative (e.g., estrogen-receptor and progesterone-receptor negative) and HER2 positive. CTCs of normal-like breast cancer can be hormone-receptor positive (e.g., estrogen-receptor and/or progesterone-receptor positive), HER2 negative, and with low levels of the protein Ki-67.

In some embodiments, the diseased cells as disclosed herein can be cancer cells. Non-limiting examples of cancer cells can include cells of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, and Wilms' tumor.

In some embodiments, the CTC as detected or identified as disclosed herein is associated with a solid tumor, such as breask cancer. In some embodiments, the CTC as detected or identified as disclosed herein is associated with a blood cancer (e.g., non-solid tumor), such as leukemia, lymphoma, myelodysplastic syndromes (MDS), myeloproliferative disorder (MPD), and multiple myeloma.

In some embodiments, the method disclosed herein can scan a plurality of cells (e.g., millions of cells) from the blood of a subject and acquire one or more 3-dimensional cell images per cell, with resolution comparable to that of confocal microscopy, thereby enhancing the accuracy of biomarker quantitation.

The method disclosed herein can be used to identify CTCs exhibiting one or more target biomarkers. A target biomarker can be a tumor antigen (or a carcinoma-associated antigen). The tumor antigen can be encoded by a gene carrying one or more mutations. Alternatively, the tumor antigen can be encoded by a gene that does not carry a mutation. The tumor antigen can be a receptor polypeptide (e.g., a cell surface receptor polypeptide). The tumor antigen can be an ion channel, such as a cationic ion channel for calcium signaling in a cell. In some embodiments, the tumor antigen can be a calcium signal transducer, such as Tumor-associated calcium signal transducer 2 (Trop2). In some embodiments, the tumor antigen is not EpCAM, Vimentin (Vim), and/or Cytokeratin (CK).

CTC assessment can be a way of identifying more aggressive components of tumors. By sequencing the tumor genome in patients with metastatic breast cancer and enumerating and characterizing the CTCs present, genetic alterations that could result in higher levels of more aggressive CTCs can be identified. Additionally, if an actionable genetic alteration is found, a targeted therapy could be used in treatment with continued follow-up of CTCs over time.

Multiplex testing (e.g., 10 antibodies on a single cell) can enhance detection and profiling heterogeneity of circulating tumor cells. The counting process can be automated.

### Examples

### Example 1. Probe Components and Manufacturing

The immunostaining reagents can include two components:
1. Labeling moieties (e.g., immunofluorescent probes in which antibodies that are linked to a fluorochrome via a specifically designed, synthetic polypeptide linker, such as a DNA oligomer).
2. A cleavage moiety cocktail (e.g., a fluorochrome cleaving cocktail that utilizes one or more enzymes, such as the CRISPR/Cas system (e.g., CRISPR/Cas9 system) programmed to recognize the oligomer sequence and releases the fluorochrome attached to the antibody).

### Example 2. Generation of a Labeling Moiety (e.g., MultiFluor Probe Manufacturing)

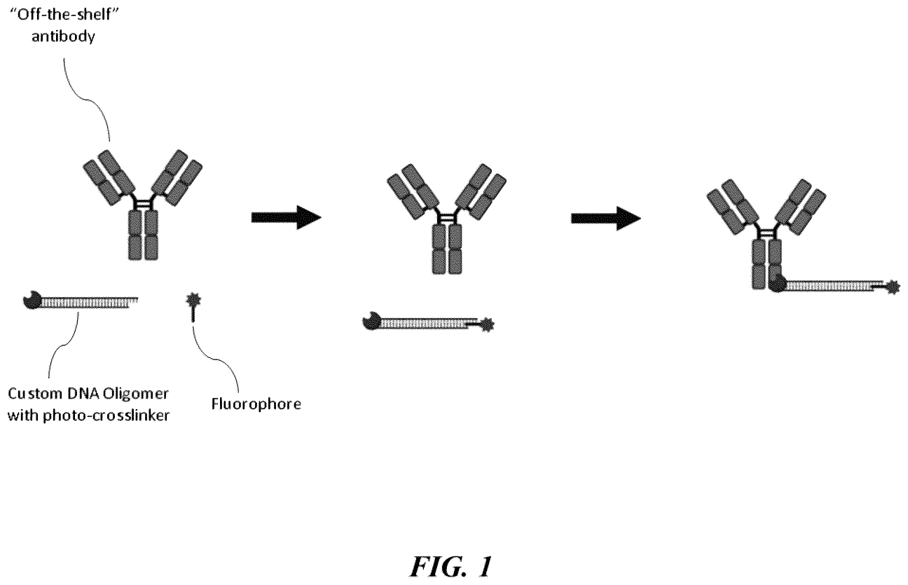

FIG. 1 schematically illustrates generation of a labeling moiety, for example, via (i) functionalizing a primary (or off-the-shelf) antibody with a detactable tag (e.g., a fluorophore) via a polypeptide linker (e.g., a custom DNA oligomer with a photo-crosslinker, wherein the photo-crosslinker is for coupling to the primary antibody).

Antibody labeling reagents, that allow site-specific and covalently couple a DNA oligomer with the Fc region of various off-the-shelf antibodies, can be used. For example, oYo Link reagents contain low molecular weight, high-affinity antibody-binding domains embedding a photo-crosslinker within their Fc-binding site. Upon illumination with non-damaging 365 light, oYo-Link forms a covalent bond with the antibody (Light-Activated Site-Specific Conjugation (LASIC)). This site-specific antibody labeling ensures that the label does not interfere with antigen binding with the target antigen.

In the design of the oligo oYo-Link sequence, further signal amplification is possible using a DNA labeling kit. For example, in a two-step process using the DNA labeling kit, the aminoallyl dUTP is enzymatically incorporated by polymerase and then a reactive fluorophore is used to label the incorporated aminoallyl group. The custom oligo can include guanine-rich repeats on the end of the oligo closest to the fluorophore so that the oligo can be used for labeling and signal amplification.

### Example 3. Analysis of a Biological Sample Using the Labeling Moiety and the Cleavage Moiety (e.g., MultiFluor Staining Protocol)

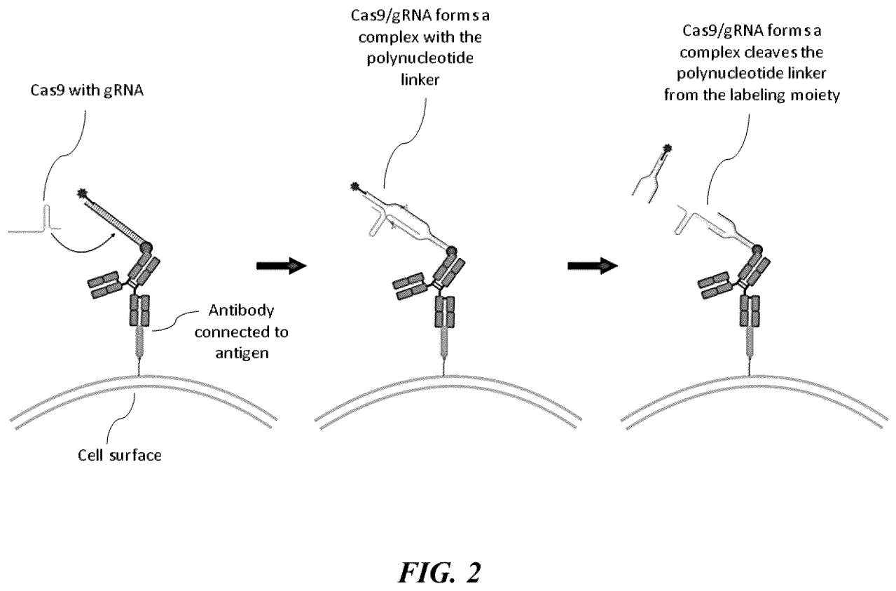

FIG. 2 schematically illustrates an example process of using the labeling moiety and the cleavage moiety, as described herein, for imaging a biological sample, e.g., a cell. The example process can comprise the following steps:
1. Generate DNA oligomer with custom sequence.
2. Generate CRISPR-Cas9 guide RNAs (gRNA) targeting the DNA oligomer sequence.
3. Create oYo-Link oligo coupled antibodies.
Utilizing the oYo-Link Oligo custom kit conjugate DNA oligomer with custom antibody. This construct can be linked to (e.g., covalently conjugated to via using a polynucleotide linker as disclosed herein) a fluorophore, such as HyperBright 488, 647, Alexa Fluor 488, 647 etc.
Attach the oYo-Link to Fc region of selected antibodies.
   a. Potential fluorescence amplification can be implemented using DNA labels.
4. Mix a first cocktail of probes and contact immobilized cell suspension
5. Image immobilized cells.
6. Process with CRISPR-Cas9 reagents to cleave oYo-link oligomers and make cells available for staining with a follow-up staining with probes and imaging of previously identified target cells.

### Embodiments

The following non-limiting embodiments provide illustrative examples of the invention, but do not limit the scope of the invention.

Embodiment 1. A method for analyzing a biological sample, the method comprising:
(a) contacting the biological sample with a labeling moiety, wherein the labeling moiety comprises a binding moiety that is coupled to a detectable tag via a polynucleotide linker, wherein the binding moiety exhibits specific binding to a target ligand;
(b) subsequent to (a), imaging the biological sample to obtain an image, wherein the image is indicative of presence or absence of the target ligand in the biological sample based on staining or lack of staining by the labeling moiety; and
(c) subsequent to (b), contacting the biological sample with a cleavage moiety, wherein the cleavage moiety forms a complex with the polynucleotide linker, wherein, after formation of the complex, the cleavage moiety effects cleavage of the polynucleotide linker to release the detectable tag from the binding moiety,
optionally wherein:
   (1) the method further comprises, subsequent to (c), repeating (a) and (b) with an additional labeling moiety, wherein the additional labeling moiety comprises an additional binding moiety, wherein the additional binding moiety exhibits specific binding to an additional target ligand that is different from the target ligand;
   (2) the method further comprises, subsequent to (c), repeating (a), (b), and (c) with the additional labeling moiety;
   (3) (a) further comprises contacting the biological sample with an additional labeling moiety, wherein the additional labeling moiety comprises an additional binding moiety that is coupled to an additional detectable tag via an additional polynucleotide linker, wherein (i) the additional binding moiety exhibits specific binding to an additional target ligand that is different from the target ligand and (ii) the additional detectable tag is different from the detectable tag;
      (b) further comprises imaging the biological sample to obtain an additional image, wherein the additional image is indicative of presence or absence of the additional target ligand in the biological sample based on staining or lack of staining by the additional labeling moiety; and
      (c) further comprises contacting the biological sample with an additional cleavage moiety, wherein the additional cleavage moiety forms an additional complex with the additional polynucleotide linker, wherein, after formation of the additional complex, the additional cleavage moiety effects cleavage of the additional polynucleotide linker to release the additional detectable tag from the additional binding moiety;
   (4) the polynucleotide linker and the additional polynucleotide linker are substantially the same;
   (5) the polynucleotide linker and the additional polynucleotide linker are different from each other;
   (6) the cleavage moiety is not expressed by a cell of the biological sample;
   (7) the labeling moiety is disposed at an extracellular space of a cell of the biological sample, and wherein the cleavage moiety is disposed at the extracellular space of the cell;
   (8) the target polynucleotide linker has a length of at least about 10 nucleobases;
   (9) the target polynucleotide linker has a length of at least about 50 nucleobases;
   (10) the target polynucleotide linker has a length of at least about 200 nucleobases;
   (11) (i) the cleavage moiety comprises an enzyme; and/or
      (ii) the cleavage moiety comprises a complex, wherein the complex comprises a Cas protein and a guide nucleic acid molecule, wherein the guide nucleic acid molecule exhibits specific binding to the polynucleotide linker,
         further optionally wherein the guide nucleic acid molecule does not comprise a dye;
(12) (i) the biological sample is not subjected to enrichment for the diseased cell prior to (b); or
   (ii) the biological sample is subjected to enrichment for the diseased cell prior to (b);
(13) the method further comprises, subsequent to (c), (d) analyzing the image to identify a diseased cell from the biological sample;
(14) the biological sample comprises a cell, further optionally wherein the cell is a circulating tumor cell (CTC) or a lymphocyte;
(15) the imaging in (b) comprises selective plane imaging microscopy, and wherein the image is a planar image;
(16) the imaging comprises scanning the biological sample with a laser light sheet;
(17) the binding moiety is covalently coupled to the detectable tag via the polynucleotide linker;
(18) the binding moiety comprises an antibody or an antigen-binding fragment thereof;
(19) the detectable tag is a dye;
(20) the polypeptide linker is a single-stranded nucleic acid molecule; and/or
(21) the polypeptide linker is a double-stranded nucleic acid molecule.

Embodiment 2. A system for analyzing a biological sample, the system comprising:
a chamber, wherein the chamber comprises a container for holding the biological sample;
an imaging unit optically coupled to the chamber, wherein the imaging unit is configured to image the biological sample when disposed in the container; and
a processor operatively coupled to the imaging unit, wherein the processor is configured to:
   (a) direct flow of a labeling moiety to the container, wherein the labeling moiety comprises a binding moiety that is coupled to a detectable tag via a polynucleotide linker, wherein the binding moiety exhibits specific binding to a target ligand;
   (b) subsequent to (a), direct the imaging unit to image the biological sample in the container, to obtain an image, wherein the image is indicative of presence or absence of the target ligand in the biological sample based on staining or lack of staining by the labeling moiety; and
   (c) subsequent to (b), direct flow of a cleavage moiety to the container, wherein the cleavage moiety forms a complex with the polynucleotide linker, wherein, after formation of the complex, the cleavage moiety effects cleavage of the polynucleotide linker to release the detectable tag from the binding moiety,
optionally wherein:
   (1) the system further comprises a reservoir, wherein the reservoir comprises a source of the labeling moiety, wherein the reservoir is in fluid communication with the container;
   (2) the system further comprises a reservoir, wherein the reservoir comprises a source of the cleavage moiety, wherein the reservoir is in fluid communication with the container;
   (3) the imaging unit comprises (i) a light source configured to direct a light towards the container, and (ii) a detector configured to detect the biological sample upon exposure of the biological sample to the light;
   (4) an optical axis of the light source is not parallel to an optical axis of the detector;
   (5) an optical axis of the light source is substantially perpendicular to an optical axis of the detector;
   (6) the processor is further configured to, subsequent to (c), repeating (a) and (b) with an additional labeling moiety, wherein the additional labeling moiety comprises an additional binding moiety, wherein the additional binding moiety exhibits specific binding to an additional target ligand that is different from the target ligand,
      optionally wherein the processor is further configured to, subsequent to (c), repeating (a), (b), and (c) with the additional labeling moiety;
   (7) the processor is further configured to:
      in (a), directing flow of an additional labeling moiety, wherein the additional labeling moiety comprises an additional binding moiety that is coupled to an additional detectable tag via an additional polynucleotide linker, wherein (i) the additional binding moiety exhibits specific binding to an additional target ligand that is different from the target ligand and (ii) the additional detectable tag is different from the detectable tag;
      in (b), direct the imaging unit to image the biological sample in the container, to obtain an additional image, wherein the additional image is indicative of presence or absence of the additional target ligand in the biological sample based on staining or lack of staining by the additional labeling moiety; and
      in (c), directing the flow of an additional cleavage moiety, wherein the additional cleavage moiety forms an additional complex with the additional polynucleotide linker, wherein, after formation of the additional complex, the additional cleavage moiety effects cleavage of the additional polynucleotide linker to release the additional detectable tag from the additional binding moiety;
   (8) the polynucleotide linker and the additional polynucleotide linker are substantially the same, optionally wherein the polynucleotide linker and the additional polynucleotide linker are different from each other;
   (9) the cleavage moiety is not expressed by a cell of the biological sample;
   (10) the labeling moiety is disposed at an extracellular space of a cell of the biological sample, and wherein the cleavage moiety is disposed at the extracellular space of the cell;
   (11) the target polynucleotide linker has a length of at least about 10 nucleobases;
   (12) the target polynucleotide linker has a length of at least about 50 nucleobases;
   (13) the target polynucleotide linker has a length of at least about 200 nucleobases;
   (14) the cleavage moiety comprises an enzyme;
   (15) the cleavage moiety comprises a complex, wherein the complex comprises a Cas protein and a guide nucleic acid molecule, wherein the guide nucleic acid molecule exhibits specific binding to the polynucleotide linker,
      optionally wherein the guide nucleic acid molecule does not comprise a dye;
   (16) (i) the biological sample is not subjected to enrichment for the diseased cell prior to (b);
      (ii) the biological sample is subjected to enrichment for the diseased cell prior to (b);
   (17) the processor is further configured to, subsequent to (c), (d) analyze the image to identify a diseased cell from the biological sample;
   (18) the biological sample comprises a cell,
      optionally wherein the cell is a circulating tumor cell (CTC) or a lymphocyte;
   (19) imaging the biological sample comprises selective plane imaging microscopy, and wherein the image is a planar image;
   (20) imaging the biological sample comprises scanning the biological sample with a laser light sheet;
   (21) the binding moiety is covalently coupled to the detectable tag via the polynucleotide linker;
   (22) the binding moiety comprises an antibody or an antigen-binding fragment thereof;
   (23) the detectable tag is a dye;
   (24) the polypeptide linker is a single-stranded nucleic acid molecule; and/or
   (25) the polypeptide linker is a double-stranded nucleic acid molecule.

1. A method for analyzing a biological sample, the method comprising:
(a) contacting the biological sample with a labeling moiety, wherein the labeling moiety comprises a binding moiety that is coupled to a detectable tag via a polynucleotide linker, wherein the binding moiety exhibits specific binding to a target ligand;
(b) subsequent to (a), imaging the biological sample to obtain an image, wherein the image is indicative of presence or absence of the target ligand in the biological sample based on staining or lack of staining by the labeling moiety; and
(c) subsequent to (b), contacting the biological sample with a cleavage moiety, wherein the cleavage moiety forms a complex with the polynucleotide linker, wherein, after formation of the complex, the cleavage moiety effects cleavage of the polynucleotide linker to release the detectable tag from the binding moiety.

2. The method of claim 1, further comprising, subsequent to (c), repeating (a) and (b) with an additional labeling moiety, wherein the additional labeling moiety comprises an additional binding moiety, wherein the additional binding moiety exhibits specific binding to an additional target ligand that is different from the target ligand.

3. The method of claim 1, further comprising, subsequent to (c), repeating (a), (b), and (c) with the additional labeling moiety.

4. The method of claim 1, wherein:
(a) further comprises contacting the biological sample with an additional labeling moiety, wherein the additional labeling moiety comprises an additional binding moiety that is coupled to an additional detectable tag via an additional polynucleotide linker, wherein (i) the additional binding moiety exhibits specific binding to an additional target ligand that is different from the target ligand and (ii) the additional detectable tag is different from the detectable tag;
(b) further comprises imaging the biological sample to obtain an additional image, wherein the additional image is indicative of presence or absence of the additional target ligand in the biological sample based on staining or lack of staining by the additional labeling moiety; and
(c) further comprises contacting the biological sample with an additional cleavage moiety, wherein the additional cleavage moiety forms an additional complex with the additional polynucleotide linker, wherein, after formation of the additional complex, the additional cleavage moiety effects cleavage of the additional polynucleotide linker to release the additional detectable tag from the additional binding moiety.

5. (canceled)

6. (canceled)

7. The method of claim 1, wherein the cleavage moiety is not expressed by a cell of the biological sample.

8. The method of claim 1, wherein the labeling moiety is disposed at an extracellular space of a cell of the biological sample, and wherein the cleavage moiety is disposed at the extracellular space of the cell.

9. The method of claim 1, wherein the target polynucleotide linker has a length of at least about 10 nucleobases.

10. (canceled)

11. (canceled)

12. The method of claim 1, wherein the cleavage moiety comprises an enzyme.

13. The method of claim 1, wherein the cleavage moiety comprises a complex, wherein the complex comprises a Cas protein and a guide nucleic acid molecule, wherein the guide nucleic acid molecule exhibits specific binding to the polynucleotide linker.

14. The method of claim 13, wherein the guide nucleic acid molecule does not comprise a dye.

15. The method of claim 1, wherein the biological sample is not subjected to enrichment for the diseased cell prior to (b).

16. The method of claim 1, further comprising, subsequent to (c), (d) analyzing the image to identify a diseased cell from the biological sample.

17. The method of claim 1, wherein the biological sample comprises a cell.

18. The method of claim 17, wherein the cell is a circulating tumor cell (CTC).

19. The method of claim 1, wherein the imaging in (b) comprises selective plane imaging microscopy, and wherein the image is a planar image.

20. The method of claim 1, wherein the imaging comprises scanning the biological sample with a laser light sheet.

21. The method of claim 1, wherein the binding moiety is covalently coupled to the detectable tag via the polynucleotide linker.

22. The method of claim 1, wherein the binding moiety comprises an antibody or an antigen-binding fragment thereof.

23. (canceled)

24. The method of claim 1, wherein the polypeptide linker is a single-stranded nucleic acid molecule.

25. The method of claim 1, wherein the polypeptide linker is a double-stranded nucleic acid molecule.

26.-55. (canceled)
